(19) United States
(12) Patent Application Publication
(10) Pub. No.: US 2013/0149344 A1
(43) Pub. Date: Jun. 13, 2013

(54) Pleasure Enhancing Condom

(71) Applicant: Arthur Ruppin, Ringwood, NJ (US)

(72) Inventor: Arthur Ruppin, Ringwood, NJ (US)

(21) Appl. No.: 13/324,950

(22) Filed: Dec. 13, 2011

(51) Int. Cl. A61K 9/00; A61P 15/16; B05D 1/18; A61K 31/568

(52) U.S. Cl. 424/400; 514/178; 427/430.1

(57) ABSTRACT

A pleasure enhancing condom comprises a tubular member being configured to encase an organ being operable for penetration and or contact during a sexual activity. A chemical composition is disposed about at least a portion of the exterior surface of the tubular member. The chemical composition comprises one or more hormones being operable for transferring to a contacting individual to enhance the sexual activity.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to sexual intercourse devices. More particularly, the invention relates to pleasure enhancing condoms.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

A study performed at Tel Aviv University Medical School has shown that reception of estrogens and/or testosterones hormones for a certain percentage of women during vaginal sexual intercourse enhances the pleasure of the women as compared to performing sexual intercourse without receiving such hormones. Furthermore, it is known that during orgasm associated with sexual intercourse, large amounts of endorphins are released in women and in men.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an example condom, in accordance with an embodiment of the present invention;

FIG. 2 illustrates an example condom as described with reference to FIG. 1 which has been extended, in accordance with an embodiment of the present invention;

FIG. 3 illustrates an example cross-sectional view for the condom as described with reference to FIG. 2, in accordance with an embodiment of the present invention;

FIG. 4 illustrates an example cross-sectional view for the condom as described with reference to FIG. 2, in accordance with an embodiment of the present invention;

FIGS. 5A-D illustrate an example apparatus for fabricating condoms as described with reference to FIGS. 1-4, in accordance with an embodiment of the present invention; and

FIG. 6 illustrates an example method for fabricating a condom, in accordance with an embodiment of the present invention.

Unless otherwise indicated illustrations in the figures are not necessarily drawn to scale.

### DETAILED DESCRIPTION OF SOME EMBODIMENTS

Embodiments of the present invention are best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Embodiments of the present invention will be described which provides means and methods for a pleasure enhancing condom. The condom may be constructed of any known material. Non-limiting examples of materials for construction include latex, polyurethane and rubber. The condom is coated, laced or embedded in whole or in partial with a chemical composition. As a non-limiting example, chemical composition may be a hormone. Non-limiting examples of hormones include estrogens, testosterones and endorphins. Chemical composition provides increased pleasure to one or more persons participating in sexual intercourse. As a non-limiting example, increased pleasure may be provided to women during vaginal intercourse. Furthermore, condom aids in providing protection from pregnancy and transmission of diseases. During sexual intercourse where a person is being penetrated by an organ such as, but not limited to, a body organ, dildo, vibrator, etc., covered by the condom, contact between the condom and the penetrated person's body causes the chemical composition or a portion of the chemical composition to be transferred to the penetrated person. The transference followed by absorption or receipt of the chemical composition by the receiving person provides increased pleasure from sexual intercourse. Non-limiting examples of chemicals associated with the chemical composition for the condom include lubricants, spermicidals, anti-bacterials, anti-virals and flavor materials.

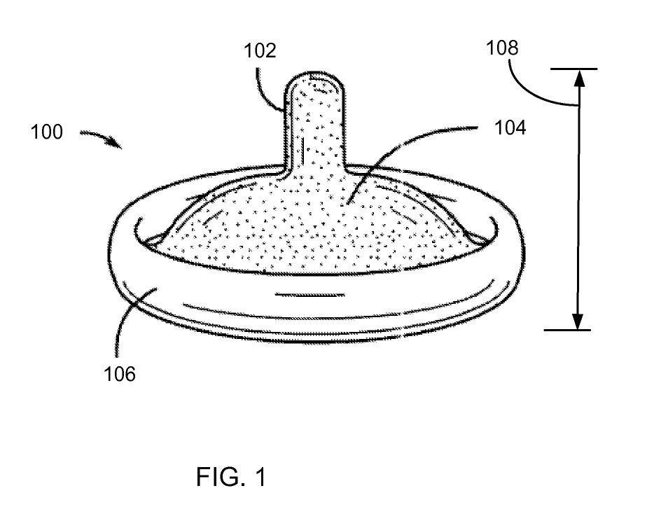

FIG. 1 illustrates an example condom, in accordance with an embodiment of the present invention.

A condom 100 includes a tip 102, a chemical composition 104 and a ring 106.

Condom 100 is used during the act of sexual intercourse for preventing pregnancy and or prevention of transmission of diseases. Furthermore, condom 100 provides enhanced sexual pleasure to a first person.

Tip 102 is used for storage of sperm and/or semen following ejaculation by a second person.

Chemical composition 104 is used for enhancing pleasure of a first person (not shown) receiving chemical composition 104. As a non-limiting example, chemical composition 104 may be configured of hormones. Non-limiting examples of hormones include estrogens, testosterones or endorphins. Furthermore, hormones may be configured of any known mixture and concentration of estrogens, testosterones and/or endorphins. The preferred range is between 0% and 200% of the concentration range of the hormones found in semen, as to each specific hormone and the maximum range shall be between 0% and 1000% of the concentration range of the hormones found in semen, as to each specific hormone and or for the total hormones.

Non-limiting examples for the form factor of the chemical composition include gel, paste, liquid, powder or solid. The chemical composition may be configured such that it may be transferred from condom 100 to first person for absorption or receipt by first person. Furthermore, absorption or receipt may be induced via friction and or contact generated between condom 100 and first person. As a non-limiting example, chemical composition 104 may be absorbed by first person's vagina or other body cavity including and not limited to the anus and or mouth.

Chemical composition 104 may be provided by any known means. As a non-limiting example, chemical composition 104 may be stored in one or more layers associated with the external surface of condom 100. Chemical composition 104 may be provided via a coating of chemical composition 104 located on the external surface of condom 100. Furthermore, a portion of chemical composition 104 may be released from condom 100 upon contact with penetrated opening or cavity.

Ring 106 is used for storage or rolling of condom in a compact form and for providing support and retention during use. Non-limiting examples for materials configured for ring 106, which is customarily, but not necessarily an integral part of condom 100, include latex, polyurethane, rubber, calfskin, natural skin and/or artificial skin.

Condom has a non-extended length as noted by a non-extended length 108.

In operation, tip is squeezed to remove air and condom 100 is slipped over the organ (e.g. penis) of a second person (not shown) with ring 106 being unrolled for encapsulating the organ. Condom 100 is inserted into cavity of first person by second person during sexual intercourse. Chemical composition 104 is located on the outer surface of condom 100 or is transferred or traverses to the outer surface of condom 100. Friction and or contact during penetration between the outer surface of the condom and the penetrated opening causes chemical composition 104 to be transferred from condom 100 to first person's body. Chemical composition 104 is absorbed by body of first person for producing enhanced pleasure. As a non-limiting example, chemical composition 104 may be absorbed or received via the vagina of first person. Following ejaculation by second person, sperm/semen collects in volume of tip 102. Following sexual intercourse, condom 100 is discarded.

FIG. 1 illustrates an example condom where tip of condom is squeezed during application, condom covers penis, condom is inserted, chemical composition is transferred, chemical composition is absorbed or received, chemical composition provides enhanced pleasure, sperm/semen is collected in tip following ejaculation and condom is discarded following sexual intercourse, in accordance with an embodiment of the present invention.

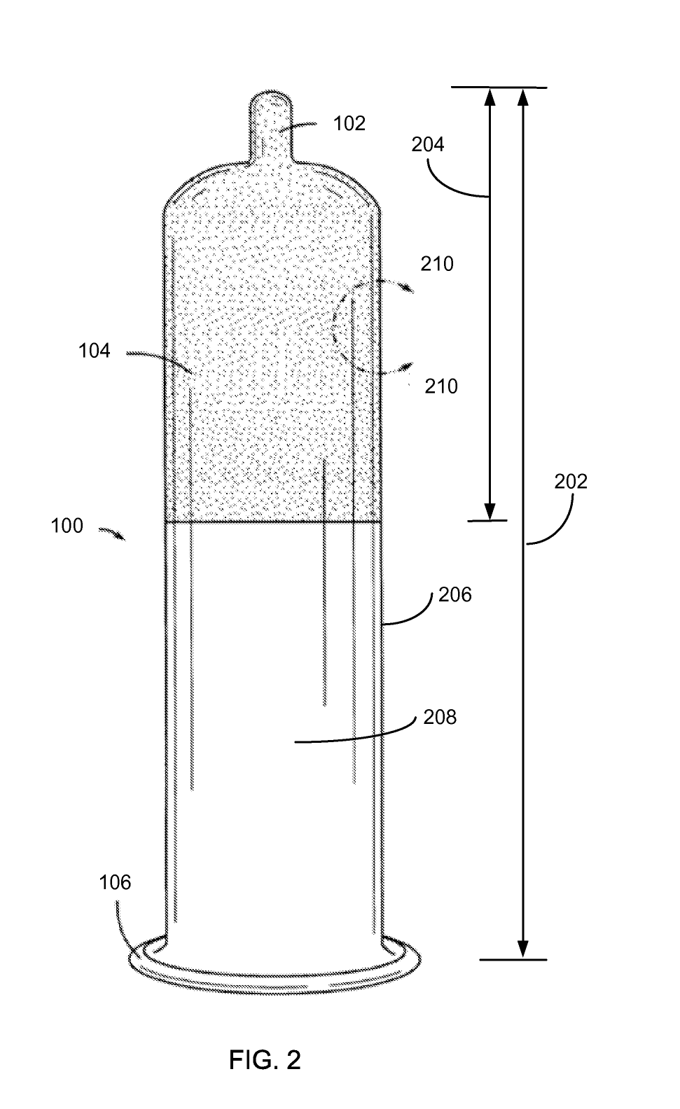

FIG. 2 illustrates an example condom as described with reference to FIG. 1 which has been extended, in accordance with an embodiment of the present invention.

Condom 100 has an extended length as noted by an extended length 202 and is larger than non-extended length 108 as described with reference to FIG. 1.

A portion of condom with a length as noted by a chemical composition length 204 supplies chemical composition 104. In some embodiments, chemical composition length 204 may be equal to extended length 202 or any portion of extended length 202.

Extending condom 100 as illustrated in FIG. 2 enables viewing a body portion 206 of condom 100 not presented as viewable as described with reference to FIG. 1 as body portion 206 is rolled-up and encapsulated within ring 106 as shown in FIG. 1. Non-limiting examples for materials configured for body portion 206 include latex, polyurethane, rubber, calfskin, natural skin and/or artificial skin.

A cavity 208 is located internally to condom 100 for encapsulating an organ such as, but not limited to, penis, finger, dildo vibrator, etc.

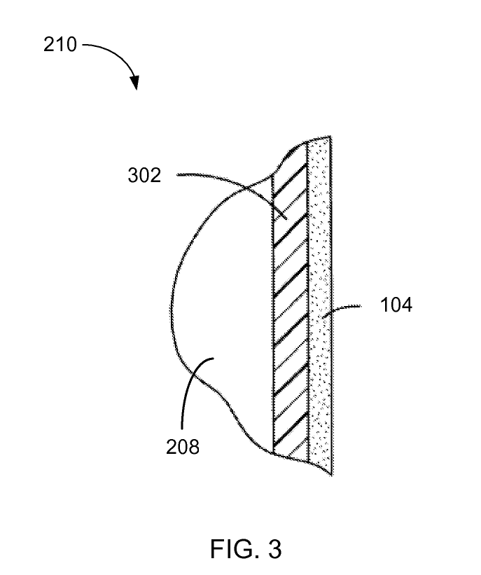

A cut-out portion 210 provides for a more detailed view as described in the paragraphs below with respect to FIG. 3.

FIG. 2 illustrates an example condom as described with reference to FIG. 1 which has been extended where a body portion of the condom is visible.

FIG. 3 illustrates an example cross-sectional view for the condom as described with reference to FIG. 2, in accordance with an embodiment of the present invention.

Cut-out portion 210 presents chemical composition 104 located external to a condom wall 302. Furthermore, cavity 208 is encapsulated within condom wall 302. Non-limiting examples for materials configured for condom wall 302 include latex, polyurethane, rubber, calfskin, natural skin and/or artificial skin.

Chemical composition 104 is attached to condom wall 302. Furthermore, chemical composition 104 is transferred to and absorbed or contacted by first person during sexual intercourse. Condom wall 302 aids in preventing pregnancy of first person and provides protection from transmission of disease to first and second person.

FIG. 3 illustrates an example cross-sectional view for the condom as described with reference to FIG. 2 where a chemical composition is attached to the exterior wall of a condom for enhancing pleasure during sexual intercourse.

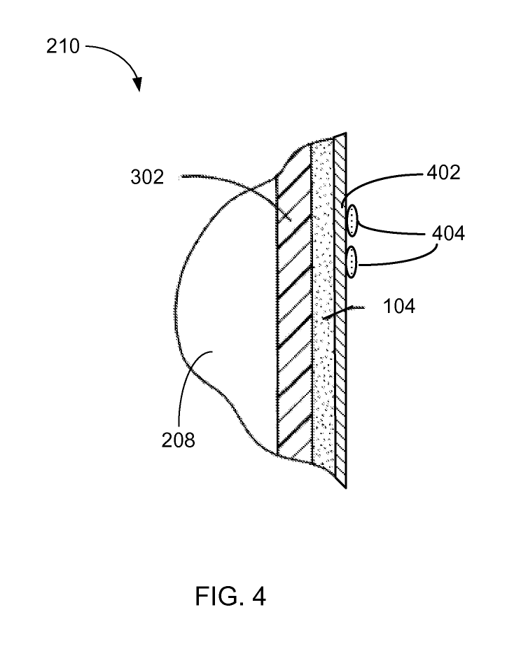

FIG. 4 illustrates an example cross-sectional view for the condom as described with reference to FIG. 2, in accordance with an embodiment of the present invention.

Cut-out portion 210 presents chemical composition 104 located external to condom wall 302 and encapsulated within a permeable membrane 402. Furthermore, cavity 208 is encapsulated within condom wall 302. Permeable membrane 402 enables the traversing of chemical composition 104 to the external wall of permeable membrane 402 as noted by an exterior chemical composition 404.

Chemical composition 104 resides between condom wall 302 and permeable membrane 402. Chemical composition 104 traverses permeable membrane 402 during sexual intercourse to form exterior chemical composition 404. Furthermore, exterior chemical composition 404 is transferred to and absorbed or received by first person during sexual intercourse. Absorbed or received exterior chemical composition 404 provides enhanced pleasure during sexual intercourse. Condom wall 302 aids in preventing pregnancy of first person and provides protection from transmission of disease to first and second person.

FIG. 4 illustrates an example cross-sectional view for the condom as described with reference to FIG. 2 where a permeable membrane provides retention of chemical composition prior to sexual intercourse and enables traversing of the chemical composition during sexual intercourse.

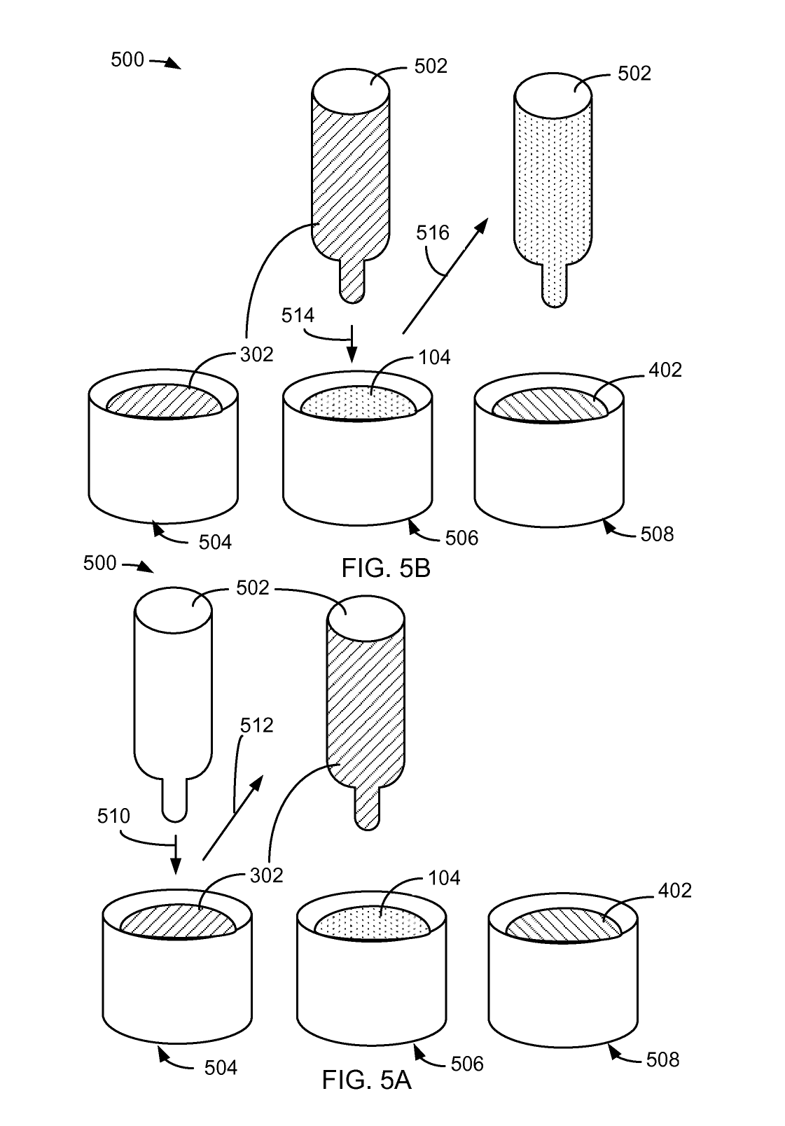

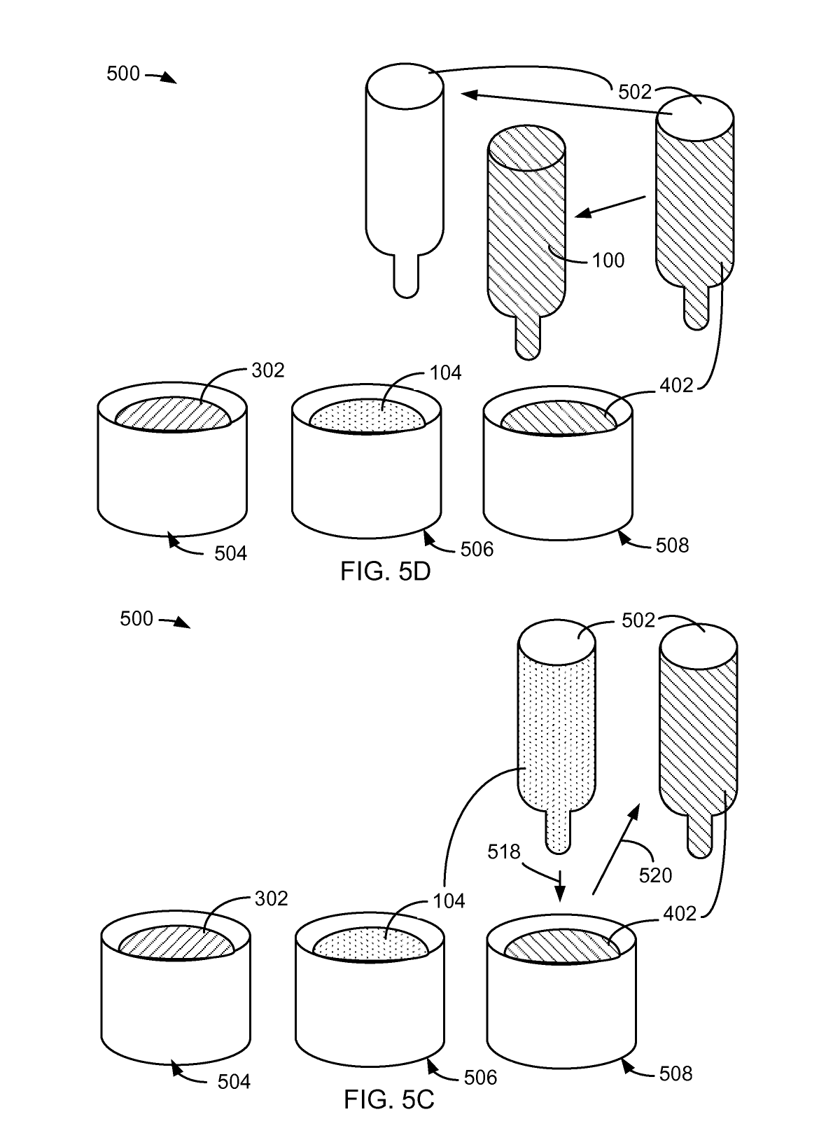

FIG. 5A illustrates an example apparatus for fabricating the condoms as described with reference to FIGS. 1-4, in accordance with an embodiment of the present invention.

An apparatus 500 includes a condom model 502, a vat 504, a vat 506 and a vat 508.

Condom model 502 provides a mechanism for shaping a condom.

Vat 504 contains a liquid version of condom wall 302 as described with reference to FIG. 3.

Vat 506 contains chemical composition 104 as described with reference to FIG. 1.

Vat 508 contains a liquid version of permeable membrane 402 as described with reference to FIG. 4.

In operation, condom model 502 is dipped into the liquid version of condom wall 302 in a downward direction 510. After condom model 502 is soaked in liquid version of condom wall 302 contained within vat 504, condom model 502 encapsulated with condom wall 302 is removed from vat 504 in an upward direction 512.

FIG. 5A illustrates an example apparatus for fabricating the condoms as described with reference to FIGS. 1-4 where a condom mold is inserted into a vat of liquid condom wall material and then removed such that the mold is covered by the condom wall material.

FIG. 5B illustrates an example apparatus for fabricating the condoms as described with reference to FIGS. 1-4, in accordance with an embodiment of the present invention.

In operation, the condom coated with condom wall material as described with reference to FIG. 5A, is dipped in a downward direction 514 into the chemical composition located within vat 506. After condom model 502 coated with condom material is soaked in chemical composition 104, condom model encapsulated with condom wall 302 and chemical composition 104 is removed from vat 506 in an upward direction 516. The condom 100 removed from permeable membrane 402 is described with reference to FIG. 3.

FIG. 5B illustrates an example apparatus for fabricating the condoms as described with reference to FIGS. 1-4 where a condom model coated with condom wall material is dipped into and removed from the chemical composition.

FIG. 5C illustrates an example apparatus for fabricating the condoms as described with reference to FIGS. 1-4, in accordance with an embodiment of the present invention.

In operation, condom model 502 coated with condom wall 302 and chemical composition 104 is dipped in a downward direction 518 into vat 508 containing a liquid version of permeable membrane 402. After soaking in the liquid version of permeable membrane 402, condom model 502 coated with condom wall 302, chemical composition 104 and permeable membrane 402 is removed in an upward direction 520. The condom 100 removed from permeable membrane 402 is described with reference to FIG. 4.

FIG. 5C illustrates an example apparatus for fabricating the condoms as described with reference to FIGS. 1-4 where a condom model coated with condom wall material and chemical composition is dipped into and removed from liquid permeable membrane material.

FIG. 5D illustrates an example apparatus for fabricating the condoms as described with reference to FIGS. 1-4, in accordance with an embodiment of the present invention.

In operation, condom 100 is removed from condom model 502.

FIG. 5D illustrates an example apparatus for fabricating the condoms as described with reference to FIGS. 1-4 where a condom is removed from a condom model.

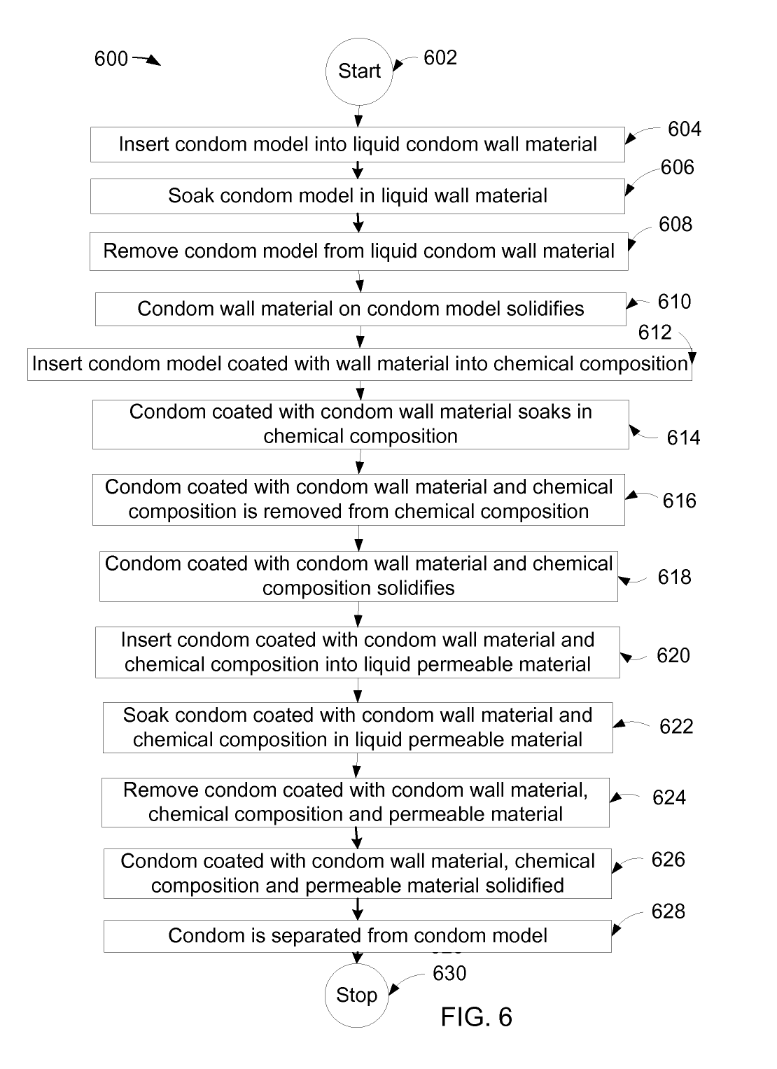

FIG. 6 illustrates an example method for fabricating a condom, in accordance with an embodiment of the present invention.

A flow chart 600 initiates in a step 602.

Then in a step 604, condom model 502 is inserted into vat 504 containing liquid condom wall 302 as described with reference to FIG. 5A.

Referring back to FIG. 6, then in a step 606 condom model 502 soaks in vat 504 containing liquid condom wall 302 as described with reference to FIG. 5A.

Referring back to FIG. 6, then in a step 608 condom model 502 coated with condom wall material is removed from vat 504 containing liquid condom wall 302 as described with reference to FIG. 5A.

Referring back to FIG. 6, then in a step 610 condom model 502 is processed for solidifying condom wall material coated on condom model 502. Non-limiting examples for processing include drying and cooling.

Referring back to FIG. 6, then in a step 612 condom model coated with condom wall 302 is inserted into vat 506 containing chemical composition 104 as described with reference to FIG. 5B.

Referring back to FIG. 6, then in a step 614 condom model 502 coated with condom wall 302 soaks in vat 506 containing chemical composition 104 as described with reference to FIG. 5B.

Referring back to FIG. 6, then in a step 616 condom model 502 coated with condom wall 302 and chemical composition 104 is removed from vat 506 containing chemical composition 104 as described with reference to FIG. 5B.

Referring back to FIG. 6, then in a step 618 condom model 502 coated with condom wall 302 and chemical composition 104 is processed for solidification or adherence of gel, paste or liquid, as the case may be. Non-limiting examples for processing include drying and cooling. In some embodiments, steps 612-618 may be repeated several times for different concentrations and or different hormones. For condoms without permeable material, step 630 may occur directly after step 618.

Referring back to FIG. 6, then in a step 620 condom model 502 coated with condom wall 302 and chemical composition 104 is inserted into vat 508 with liquid permeable membrane 402 as described with reference to FIG. 5C.

Referring back to FIG. 6, then in a step 622 condom model 502 coated with condom wall 302 and chemical composition 104 is soaked in vat 508 containing liquid permeable membrane 402 as described with reference to FIG. 5C.

Referring back to FIG. 6, then in a step 624 condom model 502 coated with condom wall 302, chemical composition 104 and permeable membrane 402 is removed from vat 508 containing liquid permeable membrane 402 as described with reference to FIG. 5C.

Referring back to FIG. 6, then in a step 626 condom coated with condom wall 302, chemical composition 104 and permeable membrane 402 is processed for solidification as described with reference to FIG. 5C.

Referring back to FIG. 6, then in a step 628 condom 100 is separated from condom model 502 as described with reference to FIG. 5D.

Referring back to FIG. 6, then in a step 630 execution of flow chart 600 terminates.

FIG. 6 illustrates an example method for fabricating a condom with a permeable membrane where a condom model is successively dipped, soaked and removed from condom wall material, chemical composition and permeable membrane followed by removal of condom from condom model. In some embodiments the condom may have a generally tubular shape as shown in the foregoing exemplary embodiments. However, those skilled in the art, in light of the teachings of the present invention, will readily recognize that a multiplicity of alternative shapes may be instead implemented such that there is an opening an that is operable for an organ or a device to enter into the opening and generally function according to at least one teachings of the present invention. In some alternative embodiment, and depending upon the needs of the particular application, the permeable membrane aspect of the foregoing embodiment(s) may not be implemented.

Those skilled in the art will readily recognize, in light of and in accordance with the teachings of the present invention, that any of the foregoing steps may be suitably replaced, reordered, removed and additional steps may be inserted depending upon the needs of the particular application, including and not limited to the elimination of the permeable membrane and the manufacturing steps associated with it. Moreover, the prescribed method steps of the foregoing embodiments may be implemented using any physical and/or hardware system that those skilled in the art will readily know is suitable in light of the foregoing teachings.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods for pleasure enhancing condoms according to the present invention will be apparent to those skilled in the art. The invention has been described above by way of illustration, and the specific embodiments disclosed are not intended to limit the invention to the particular forms disclosed. For example, the particular implementation of the pleasure enhancing condoms may vary depending upon the particular type material used. The pleasure enhancing condoms described in the foregoing were directed to latex implementations; however, similar techniques for polyurethane implementations of the present invention are contemplated as within the scope of the present invention. By way of further example, at least some of the foregoing embodiments were discussed in the context of certain manufacturing methods; however, the present invention is not limited to any given method of manufacture; e.g., without limitation, conventional condom manufacturing techniques may be used in certain embodiments of the present invention where appropriate, which may not include hormones in some applications. The invention is thus to cover all modifications, equivalents, and alternatives falling within the spirit and scope of the following claims.

Claim elements and steps herein may have been numbered and/or lettered solely as an aid in readability and understanding. Any such numbering and lettering in itself is not intended to and should not be taken to indicate the ordering of elements and/or steps in the claims.

The following is a listing of the currently pending claims:

1. A pleasure enhancing condom comprising:
a tubular member being configured to encase an organ being operable for penetration during a sexual activity;
a chemical composition being disposed only about a portion of an exterior length of said tubular member, said portion having a portion length less than said exterior length, said chemical composition comprising one or more hormones being operable for transferring to a contacting individual to enhance the sexual activity; and
a permeable membrane being configured to encapsulate said disposed chemical composition, said permeable membrane having a membrane length at least as long as said portion length, said permeable membrane being operable for enabling said chemical composition to traverse to an exterior surface of said permeable membrane during the sexual activity.

2. (canceled)

3. The pleasure enhancing condom as recited in claim 1, in which said tubular member further comprises a tip being operable for collecting fluids during the sexual activity.

4. The pleasure enhancing condom as recited in claim 1, in which said hormones are chosen from a group comprising estrogens, testosterones and endorphins

5. The pleasure enhancing condom as recited in claim 1, in which said chemical composition further comprises a lubricant.

6. The pleasure enhancing condom as recited in claim 1, in which said chemical composition further comprises a spermicidal material.

7. The pleasure enhancing condom as recited in claim 1, in which said chemical composition further comprises an anti-bacterial material.

8. The pleasure enhancing condom as recited in claim 1, in which said chemical composition further comprises an anti-viral material.

9. The pleasure enhancing condom as recited in claim 1, in which said chemical composition further comprises a flavor material.

10. The pleasure enhancing condom as recited in claim 1, in which said tubular member further comprises a ring about an open end.

11. The pleasure enhancing condom as recited in claim 1, in which said tubular member comprises latex, polyurethane, rubber, calfskin, natural skin or artificial skin.

12. A pleasure enhancing condom comprising:
a tubular member being configured to encase an organ being operable for penetration during a sexual activity, said tubular member comprising a tip being operable for collecting fluids during the sexual activity and a ring about an open end;
a chemical composition being disposed only about a portion of an exterior length of said tubular member, said portion having a portion length less than said exterior length, said chemical composition comprising one or more hormones, said hormones being chosen from a group comprising estrogens, testosterones and endorphins; and
a permeable membrane being configured to encapsulate said disposed chemical composition, said permeable membrane having a membrane length at least as long as said portion length, said permeable membrane being operable for enabling said chemical composition to traverse to an exterior surface of said permeable membrane during the sexual activity for transferring to a contacting individual to enhance the sexual activity.

13. The pleasure enhancing condom as recited in claim 12, in which said chemical composition further comprises a lubricant.

14. The pleasure enhancing condom as recited in claim 12, in which said chemical composition further comprises a spermicidal material.

15. The pleasure enhancing condom as recited in claim 12, in which said chemical composition further comprises an anti-bacterial material.

16. The pleasure enhancing condom as recited in claim 12, in which said chemical composition further comprises an anti-viral material.

17. The pleasure enhancing condom as recited in claim 12, in which said chemical composition further comprises a flavor material.

18. The pleasure enhancing condom as recited in claim 12, in which said tubular member comprises latex, polyurethane, rubber, calfskin, natural skin or artificial skin.

19. A method for making the pleasure enhancing condom as recited in claim 12, the method comprising the steps of:
inserting a condom model into a liquid wall material to form the tubular member;
removing the condom model from the liquid wall material to solidify the tubular member comprising the tip being operable for collecting fluids during the sexual activity;
inserting the condom model with the solidified tubular member into the chemical composition comprising one or more hormones to only coat the portion length of the exterior length of the tubular member, the hormones being chosen from a group comprising estrogens, testosterones and endorphins;
removing the condom model with the solidified tubular member coated with the chemical composition to solidify;
inserting the condom model with the solidified tubular member coated with the chemical composition into a liquid permeable material to encapsulate the chemical composition with the permeable membrane having the membrane length;
removing the condom model with the solidified tubular member coated with the chemical composition encapsulated with the permeable membrane to solidify; and
separating the pleasure enhancing condom from the condom model in which the permeable membrane is operable for enabling the chemical composition to traverse to an exterior surface of the permeable membrane during the sexual activity to a contacting individual to enhance the sexual activity.

20. The method as recited in claim 19, in which the chemical composition further comprises a lubricant, a spermicidal material, an anti-bacterial material, an anti-viral material, or a flavor material.
